(19) United States
(12) Patent Application Publication
(10) Pub. No.: US 2014/0200591 A1
(43) Pub. Date: Jul. 17, 2014

(54) CERVICAL SEALING APPARATUS

(71) Applicant: HOLOGIC, INC., Marlborough, MA (US)

(72) Inventors: Roy Sullivan, Uxbridge, MA (US); Daniel A. Beaudet, Lexington, MA (US); Robert F. Rioux, Ashland, MA (US)

(73) Assignee: HOLOGIC, INC., Marlborough, MA (US)

(21) Appl. No.: 13/739,444

(22) Filed: Jan. 11, 2013

(51) Int. Cl. A61B 17/42

(52) U.S. Cl. CPC A61B 17/42
USPC 606/119

(57) ABSTRACT

Aspects and embodiments directed to cervical seals are disclosed. In one embodiment, a cervical seal having adjustable length comprises a first portion and a second portion, each configured to receive an elongated central member, a length of the cervical seal corresponding to a distance between the first portion and the second portion. The cervical seal further comprises a membrane coupled to the first portion and the second portion and extending along the length of the cervical seal, wherein the first portion is movable from a first position along the elongated central member to a second position along the elongated central member, so as to change the length of the cervical seal and the membrane is configured so that a radial profile of the membrane increases in response to increasing the length of the cervical seal, so as to provide a conformable cervical seal.

### BACKGROUND

1. Field of the Invention

The present invention generally relates to cervical seals.

2. Description of Background

Intrauterine medical devices may be inserted through a patient's cervix to perform various tests or to apply various therapies. For example, an intrauterine device may be used to evaluate the integrity of a uterine cavity. In another example, a uterine ablation procedure may be performed by inserting a sheath through a patient's cervix and then extending an applicator through the distal end of the sheath into the uterus to apply therapy. In various trans-cervical procedures and devices, there is a need for a seal of the uterine cavity, for example to prevent fluids introduced into the uterus from flowing into the cervix.

### SUMMARY OF INVENTION

Aspects and embodiments of the present disclosure are directed to providing various cervical seals. According to one aspect, it is appreciated that there is a need for a cervical seal that conforms to the cervical anatomy, such as the cervical canal of a patient, thereby improving the quality of the seal. Furthermore, it is appreciated that the length of the cervical canal may vary per patient. Accordingly, there is a need for an adjustable length cervical seal.

According to one aspect, there is provided a cervical seal having an adjustable length, comprising a first portion configured to receive an elongated central member and a second portion configured to receive the elongated central member, a length of the cervical seal corresponding to a distance between the first portion and the second portion along the elongated central member. The cervical seal further comprises a membrane coupled to the first portion and the second portion and extending along the length of the cervical seal. The first portion is configured to be movable from a first position along the elongated central member to a second position along the elongated central member, so as to change the length of the cervical seal. The membrane is configured so that a radial profile of the membrane increases in response to increasing the length of the cervical seal, so as to provide a conformable cervical seal. The membrane is configured to lengthen in response to increasing the length of the cervical seal. In some embodiments, the membrane is configured to stretch in response to increasing the length of the cervical seal. In some embodiments, the cervical seal is further configured to lock the first portion to the elongated central member. In some embodiments, the membrane may have a variable durometer along the length of the cervical seal.

In some embodiments, the elongated central member may have a plurality of grooves configured to receive a locking portion of the first portion that is movable. In some embodiments, the plurality of grooves are arranged to provide a plurality of predetermined lengths of the cervical seal. In some embodiments, the elongated central member is coupled to at least one of a gynecological surgical device, an ablation device, a cervical device and a uterine device. In some embodiments, the second portion is affixed to the elongated central member, the length the cervical seal being adjustable in response to a movement of the first portion along the elongated central member.

In some embodiments, the membrane is further configured to contract in response to decreasing the length of the cervical seal. The membrane may be tapered along at least a portion of the length of the cervical seal, a diameter of the membrane changing along at least the portion of the length of the cervical seal. The membrane may be configured to conform to a shape of a cervical anatomy and may be configured to seal at least a portion of a cervical canal. The cervical seal may further be configured to seal at least one of an external os and an internal os of a cervix.

In some embodiments, the first portion of the cervical seal includes a flange having a diameter larger than a diameter of the second portion. The membrane may extend from an outer edge of the flange to an outer edge of the second portion. The flange may be spring-loaded. The diameter of the flange may be larger than a diameter of a cervical anatomy, the flange being configured to provide a physical reference from the cervical seal to the cervical anatomy. The second portion may include a contoured tip configured to locate an internal os of a cervix. The first portion may include a flange having a diameter larger than a diameter of an external os of the cervix so as to locate the external os, the cervical seal further being configured to provide a reference for a measurement of a cervical canal length of the cervix.

In various embodiments of the cervical seal having an adjustable length, the membrane may be one of a webbing, a sealed balloon and an inflatable balloon. The membrane may be backed by one of a conformable structure, a spring-loaded structure, a conformable foam and a spring-loaded foam. The membrane may be made of an elastic material. The first portion and the second portion may be made of one of a plastic material and a metal. The first portion, the second portion and the membrane may be made of an elastomeric material and the cervical seal may further include a rigid substructure disposed beneath at least one of the first portion and the second portion.

The membrane may have an accordion shape including a plurality of alternating inner and outer annular rims. The membrane may be tapered along at least a portion of the length of the cervical seal, a respective diameter of each annular rim of the plurality of alternating inner and outer annular rims changing along at least the portion of the length of the cervical seal.

The membrane may have a tubular shape and the cervical seal may further include a plurality of ribs disposed along a length of the membrane and protruding radially from a surface of the membrane, the plurality of ribs being pliable so as to allow deflecting. A diameter of the plurality of ribs may change along the length of the membrane. In some embodiments, the membrane may have a tubular shape and the cervical seal may further include a helical rib disposed around the membrane and along a length of the membrane. The cervical seal may further include a longitudinal rib disposed along the length of the membrane and coupled to a plurality of edges of the helical rib. The longitudinal rib may be tapered and a diameter of the plurality of edges of the helical rib may change along the length of the membrane.

According to another aspect, there is provided a cervical seal having an adjustable length, comprising a first portion configured to receive an elongated central member, a second portion configured to receive the elongated central member, a length of the cervical seal corresponding to a distance between the first portion and the second portion along the elongated central member. The cervical seal further comprises a membrane having a first end coupled to the first portion and a second end coupled to the second portion, the membrane extending along the length of the cervical seal. The first portion may be configured to be movable from a first position along the elongated central member to a second position along the elongated central member, so as to change the length of the cervical seal. The first portion having a first diameter and the second portion having a second diameter may be configured to remain unchanged in response to increasing the length of the cervical seal.

According to another aspect, there is provided a cervical seal comprising a rigid structure including a tubular portion having a diameter sized to receive an elongated member of a cervical device. The rigid structure further includes a flange protruding radially outward from a first end of the tubular portion, the flange having a diameter larger than a diameter of a second end of the tubular portion. The cervical seal further comprises an elastomeric portion covering at least a portion of the rigid structure. The tubular portion may be cylindrical and the elastomeric portion may include a membrane extending from an edge of the flange to the second end. The membrane may be at least one of a balloon membrane, a self-supporting membrane and a thin membrane supported by elasticity. The elastomeric portion may include a tapered portion extending along a length of the tubular portion.

In various embodiments of the cervical seal, the elastomeric portion may further include a plurality of ribs arranged along a length of the elastomeric portion and protruding from a surface thereof, the plurality of ribs being pliable so as to allow deflecting. A diameter of the plurality of ribs may change along the length of the elastomeric portion.

In some embodiments, the elastomeric portion may include a helical rib disposed around a length of the elastomeric portion. The elastomeric portion may further include a longitudinal rib disposed along the length of the elastomeric portion and coupled to a plurality of edges of the helical rib. The longitudinal rib may be tapered and a diameter of the plurality of edges of the helical rib may change along the length of the elastomeric portion.

The elastomeric portion may have an accordion shape including a plurality of alternating inner and outer annular rims along a length of the elastomeric portion. The elastomeric portion may include a tapered portion, a respective diameter of each annular rim of the plurality of alternating inner and outer annular rims changing along the length of the elastomeric portion. In some embodiments, the elastomeric portion may include a localized bump disposed on the tubular portion. A length of the bump along the tubular portion may be less than a length between the flange and the second end.

In some embodiments, the rigid structure may be made of one of a plastic material and a metal. The elastomeric portion may conform to a shape of a cervical anatomy and may be configured to seal at least a portion of a cervical canal. The diameter of the flange may be larger than a diameter of an external os of the cervical anatomy.

According to another aspect, there is provided a cervical seal comprising a tubular portion having a diameter sized to receive an elongated member of a cervical device, and a flange protruding outward from a first end of the tubular portion, the flange having a diameter larger than a diameter of a second end of the tubular portion. The cervical seal further comprises an elastomeric portion coupled at one end to the flange and at a second end to the second end of the tubular portion. The tubular portion may be cylindrical and the elastomeric portion may include a membrane. The membrane may be at least one of a balloon membrane, a self-supporting membrane and a thin membrane supported by elasticity. The elastomeric portion may include a tapered portion extending along a length of the tubular portion.

The elastomeric portion coupled to the flange and to the second end of the tubular portion may further include a plurality of ribs arranged along a length of the elastomeric portion and protruding from a surface thereof, the plurality of ribs being pliable so as to allow deflecting. A diameter of the plurality of ribs may change along the length of the elastomeric portion. In some embodiments, the elastomeric portion may include a helical rib disposed around a length of the elastomeric portion. The elastomeric portion may further include a longitudinal rib disposed along the length of the elastomeric portion and coupled to a plurality of edges of the helical rib. The longitudinal rib may be tapered and a diameter of the plurality of edges of the helical rib may change along the length of the elastomeric portion. The elastomeric portion may have an accordion shape including a plurality of alternating inner and outer annular rims along a length of the elastomeric portion. The elastomeric portion may include a tapered portion, a respective diameter of each annular rim of the plurality of alternating inner and outer annular rims changing along the length of the elastomeric portion. In some embodiments, the elastomeric portion may include a localized bump disposed on the tubular portion. A length of the bump along the tubular portion may be less than a length between the flange and the second end of the tubular portion. The elastomeric portion may conform to a shape of a cervical anatomy and may be configured to seal at least a portion of a cervical canal. The diameter of the flange may be larger than a diameter of an external os of the cervical anatomy.

According to another aspect, a method of sealing a cervical anatomy may be provided. In one embodiment, a method of sealing a cervical canal comprises inserting an adjustable length cervical seal into the cervical canal and increasing a length of the adjustable length cervical seal. The method may further comprise increasing a radial profile of the adjustable length cervical seal in response to increasing the length.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the disclosure. In the figures, each identical or nearly identical component that is illustrated in various figures is represented by a like numeral. For purposes of clarity, not every component may be labeled in every figure. In the figures:

FIG. 1A is a side elevation view of an exemplary embodiment of a cervical seal according to aspects of the present invention;

FIG. 1B is a cross sectional view of the cervical seal of FIG. 1A;

FIG. 2A is a side elevation view of another exemplary embodiment of a cervical seal according to aspects of the present invention;

FIG. 2B is a cross sectional view of the cervical seal of FIG. 2A;

FIG. 3A is a side elevation view of another exemplary embodiment of a cervical seal according to aspects of the present invention;

FIG. 3B is a cross sectional view of the cervical seal of FIG. 3A;

FIG. 4A is a side elevation view of another exemplary embodiment of a cervical seal according to aspects of the present invention;

FIG. 4B is a cross sectional view of the cervical seal of FIG. 4A;

FIG. 5A is a side elevation view of another exemplary embodiment of a cervical seal according to aspects of the present invention;

FIG. 5B is a cross sectional view of the cervical seal of FIG. 5A;

FIG. 6A is a side view of another exemplary embodiment of a cervical seal according to aspects of the present invention;

FIG. 6B is a perspective view of the cervical seal of FIG. 6A;

FIG. 7A is a side view of another exemplary embodiment of a cervical seal according to aspects of the present invention;

FIG. 7B is a perspective view of the cervical seal of FIG. 7A;

FIG. 8A is a side view of another exemplary embodiment of a cervical seal according to aspects of the present invention;

FIG. 8B is a side view of another embodiment of a cervical seal according to aspects of the present invention;

FIG. 9 is a perspective view of an exemplary embodiment of an intrauterine device including a cervical seal according to aspects of the present invention;

FIG. 10 is a perspective view of another exemplary embodiment of an intrauterine device including a cervical seal according to aspects of the present invention;

FIG. 11A illustrates an exemplary embodiment of an adjustable length cervical seal according to aspects of the present invention;

FIG. 11B illustrates the adjustable length cervical seal of FIG. 11A having an extended length according to aspects of the present invention;

FIG. 12A illustrates another exemplary embodiment of an adjustable length cervical seal according to aspects of the present invention;

FIG. 12B illustrates the adjustable length cervical seal of FIG. 12A having a membrane conforming to a cervical canal according to aspects of the present invention;

FIG. 13 illustrates an exemplary embodiment of a cervical seal having a membrane backed by a conformable structure according to aspects of the present invention;

FIG. 14 illustrates an exemplary embodiment of a cervical seal having a membrane backed by a spring-loaded structure according to aspects of the present invention;

FIG. 15 illustrates an exemplary embodiment of a cervical seal having a contoured tip according to aspects of the present invention;

FIG. 16 illustrates an exemplary embodiment of a cervical seal having a step profile according to aspects of the present invention;

FIG. 17 illustrates a profile of an exemplary embodiment of a cervical seal having internal ribs according to aspects of the present invention;

FIG. 18 illustrates an exemplary embodiment of a cervical seal in a first position and in a second position according to aspects of the present invention; and

FIG. 19 illustrates a flow chart of an exemplary method of sealing a cervical canal according to aspects of the present invention.

### DETAILED DESCRIPTION

Aspects and embodiments of this disclosure are directed to providing various seals configured to seal the uterine cavity at the cervix. The cervical seals may incorporate different shapes and firmnesses and may be configured to conform to the cervical anatomy of a patient and to seal at least one of the external os of the cervix, the internal os of the cervix, and at least a portion of the length of the cervical canal.

Embodiments of the cervical seals disclosed herein may be used with various intrauterine devices. In one embodiment, a cervical seal may include a flange or flat face configured to provide a physical reference point from the device to the anatomy of a patient. The physical reference may aid, for example, during a surgical procedure. The cervical seal may extend from the flange, for example, as an integral portion, a spring loaded portion, a floating or movable and lockable portion, or a combination thereof. In some embodiments, the cervical seal may include portions that are movable along an elongated central member and lockable to the elongated central member. The elongated central member may control placement of the seal at the anatomy. The movable and lockable portions may be configured to provide an adjustable length seal. In one embodiment, the cervical seal may include a first portion and a second portion, the first portion being a flange configured to provide a physical reference at the external os of the cervix and the second portion being movable along an elongated central member such that it extends to the internal os of the cervix, thereby providing an adjustable length cervical seal that may be configured to derive a measurement of the length of the cervical canal.

In some embodiments, the cervical seal may include a first portion and a second portion configured such that a thin, conformable membrane or webbing or otherwise a thin cross-section may connect one to the other, allowing a seal to be formed along the length of the membrane. The seal may further be aided by the tension in the membrane. The membrane may be at neutral pressure, or may be a sealed balloon or an inflatable balloon. In some embodiments, the membrane may be backed by a conformable foam or by a foam which is pre-loaded by a spring.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

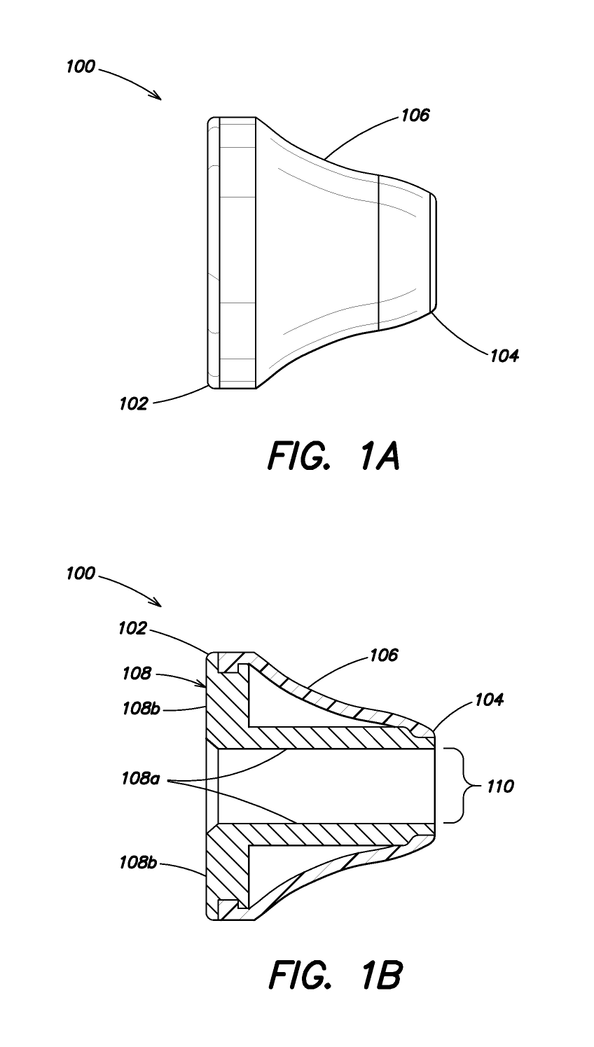

Referring to the Figures, illustrated in FIG. 1A is a cervical seal 100 having a first portion 102, a second portion 104 and a membrane 106 coupled to the first portion and the second portion. The first portion 102 and the second portion 104 form respective ends of the cervical seal 100. The first portion 102 includes a flange having a diameter larger than the diameter of the second portion of the cervical seal 100. The membrane 106 is tapered, extending from an outer edge of the first portion 102 to an outer edge of the second portion 104.

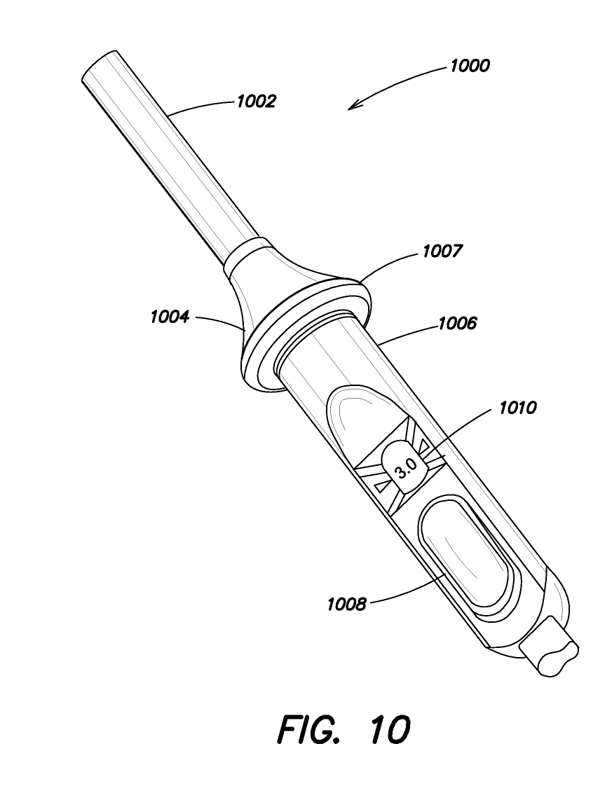

FIG. 1B shows a cross sectional view of the cervical seal 100, further illustrating a rigid structure 108. The rigid structure 108 includes a tubular portion 108a extending between the first portion 102 and the second portion 104. The tubular portion 108a is cylindrical and includes a hollow tube having a diameter 110 sized to receive an elongated member of a cervical or intrauterine device. The elongated member may be a sheath of an intrauterine device and the sheath may be inserted through the tubular portion 108a of the cervical seal 100, as shown for example in FIG. 10. The rigid structure 108 further includes a flange 108b protruding radially outward from one end of the tubular portion 108a. The first portion 102 includes the flange 108b. As shown in FIG. 1B, the membrane 106 is attached to the rigid structure 108 and extends from the first portion 102 to the second portion 104. In some embodiments, the rigid structure may be made of a plastic material or a metal. In some embodiments, the membrane may be an elastomeric membrane. In some embodiments, the membrane may be a self supporting elastomer. In various embodiments, the rigid structure may at least partially be covered by an elastomeric portion. The elastomeric portion may be a membrane, such as the membrane 106 of FIG. 1B. The membrane 106 may conform to the cervical anatomy of a patient. For example, the membrane 106 may conform to the shape of the cervical canal, thereby forming a seal of at least a portion of the cervical canal.

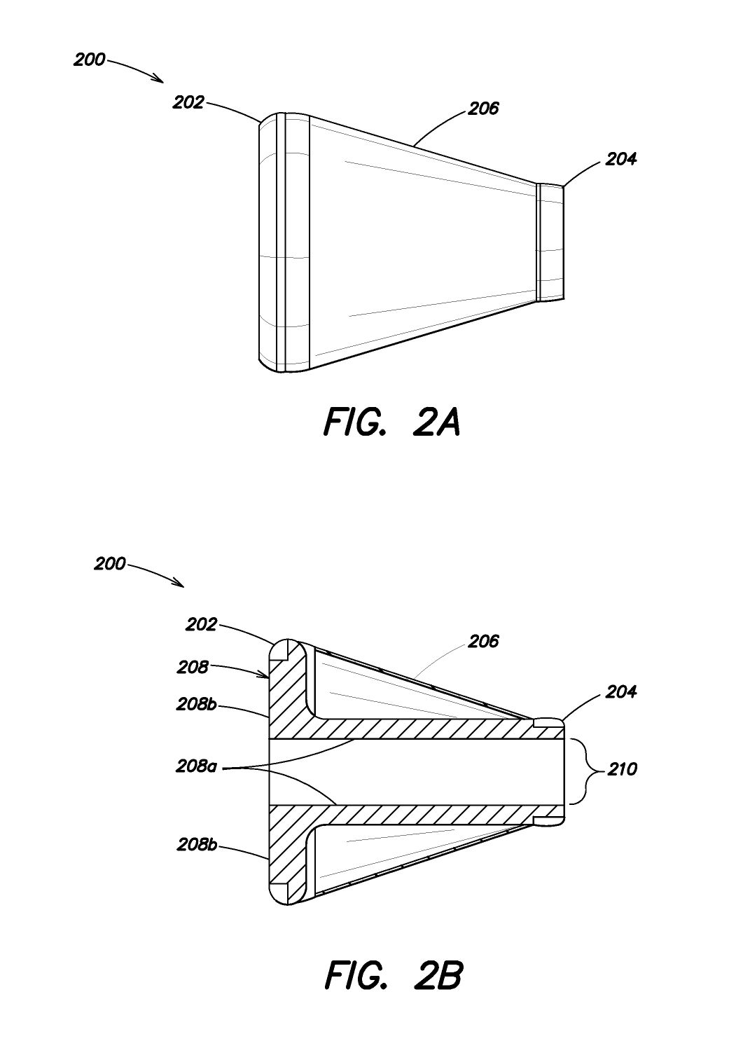

FIG. 2A illustrates a cervical seal 200 having a first portion 202, a second portion 204 and a membrane 206 coupled to the first portion and the second portion. The first portion 202 and the second portion 204 form respective ends of the cervical seal 200. The first portion 202 includes a flange having a diameter larger than a diameter of the second portion of the cervical seal 200. The membrane 206 is tapered, extending from an outer edge of the first portion 202 to an outer edge of the second portion 204. The membrane 206 is a balloon membrane. The balloon membrane may be supported by a fluid. For example, the membrane may seal a balloon and the sealed balloon may include a fluid such as air. In some embodiments, the sealed balloon may be presealed, that is sealed prior to use rather than being inflated during use. In some embodiments, the membrane 206 may include a variable durometer material, thereby allowing the membrane to take an irregular form at a desired location along the cervical seal. In one example, a distal end of the membrane 206 such as a portion of the membrane close to the second portion 204 may be more pliable relative to a proximal end of the membrane close to the first portion 202, thereby allowing the distal end of the membrane to deform more than the proximal end so as to conform to an irregular shape of a cervical anatomy.

FIG. 2B shows a cross sectional view of the cervical seal 200, further illustrating a rigid structure 208. The rigid structure 208 includes a tubular portion 208a extending between the first portion 202 and the second portion 204. The tubular portion 208a includes a hollow tube having a diameter 210 sized to receive an elongated member of a cervical or intrauterine device. The elongated member may be a sheath of an intrauterine device and the sheath may be inserted through the tubular portion 208a of the cervical seal 200, as shown for example in FIG. 10. The rigid structure 208 further includes a flange 208b protruding radially outward from one end of the tubular portion 208a. The first portion 202 includes the flange 208b. As shown in FIG. 2B, the membrane 206 is attached to the rigid structure 208 and extends from the first portion 202 to the second portion 204. As discussed above with reference to FIG. 2A, the membrane 206 is a balloon membrane, which is a thin durable membrane supported by a balloon. The balloon may be one of a sealed balloon and an inflatable balloon. In some embodiments, the thin membrane may be supported by elasticity or a cushion. In some embodiments, as shown for example in FIGS. 1A and 1B, the membrane may be a self supporting elastomer. In some embodiments, the rigid structure may be made of a plastic material or a metal. In various embodiments, the rigid structure may at least partially be covered by an elastomeric portion. The elastomeric portion may be a membrane, such as the membrane 206 of FIG. 2B. The membrane 206 may conform to the cervical anatomy of a patient. For example, the membrane 206 may conform to the shape of the cervical canal, thereby forming a seal of at least a portion of the cervical canal.

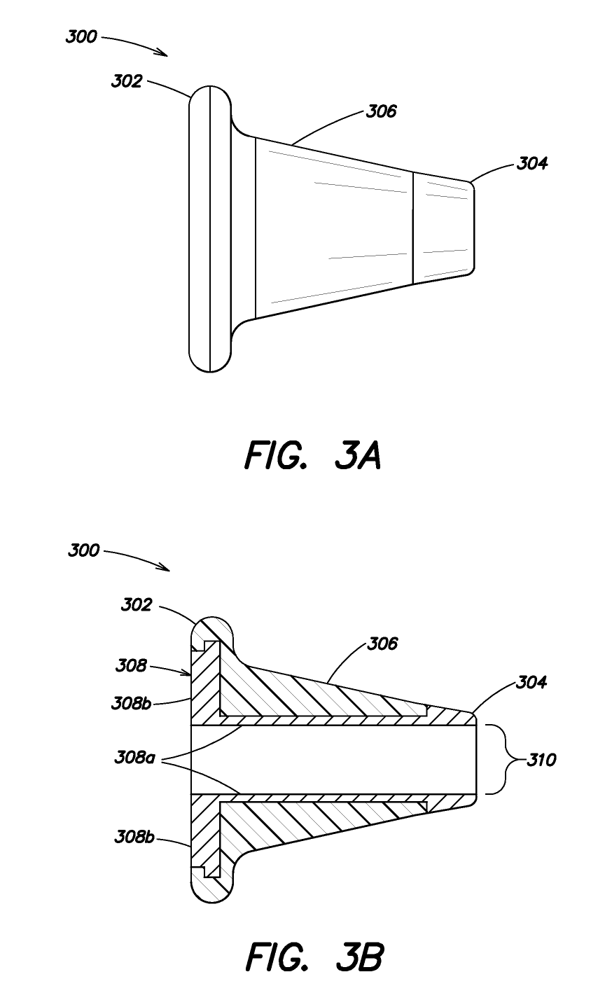

FIG. 3A illustrates another embodiment of a cervical seal 300 having a first portion 302, a second portion 304 and a dilator 306 coupled to the first portion and the second portion. The first portion 302 and the second portion 304 form respective ends of the cervical seal 300. The first portion 302 includes a flange having a diameter larger than a diameter of the second portion of the cervical seal 300. The dilator 306 is tapered, extending from an outer edge of the first portion 302 to an outer edge of the second portion 304. The dilator 306 may be used to induce dilation, that is, to expand an opening or passage such as the cervix.

FIG. 3B shows a cross sectional view of the cervical seal 300, further illustrating a rigid structure 308. The rigid structure 308 includes a tubular portion 308a extending between the first portion 302 and the second portion 304. The tubular portion 308a includes a hollow tube having a diameter 310 sized to receive an elongated member of a cervical or intrauterine device. The elongated member may be a sheath of an intrauterine device and the sheath may be inserted through the tubular portion 308a of the cervical seal 300, as shown for example in FIG. 10. The rigid structure 308 further includes a flange 308b protruding radially outward from one end of the tubular portion 308a. The first portion 302 includes the flange 308b. As shown in FIG. 3B, the dilator 306 is attached to the rigid structure 308 and extends from the first portion 302 to the second portion 304. In some embodiments, the rigid structure may be made of a plastic material or a metal. In some embodiments, the dilator may be a solid elastomeric dilator. In various embodiments, the rigid structure may at least partially be covered by an elastomeric portion. The elastomeric portion may be a dilator, such as the dilator 306 of FIG. 3B. The dilator 306 may facilitate insertion of the cervical seal 300 into a cervical canal, thereby sealing at least a portion of the cervical canal.

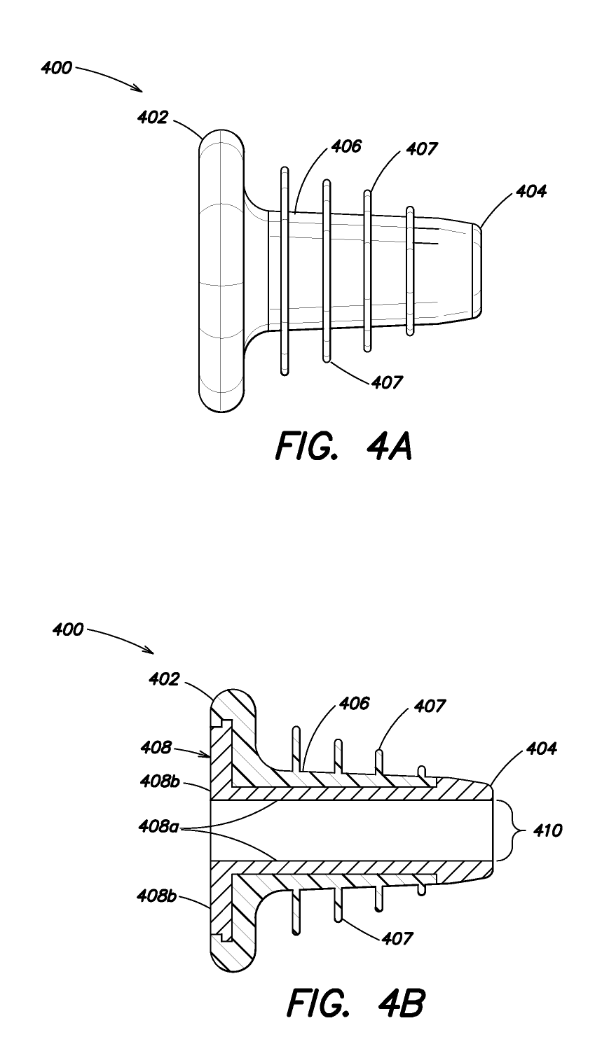

FIG. 4A illustrates another embodiment of a cervical seal 400 including a first portion 402, a second portion 404 and an elastomeric portion 406 coupled to the first portion and the second portion. The first portion 402 and the second portion 404 form respective ends of the cervical seal 400. The first portion 402 includes a flange having a diameter larger than a diameter of the second portion of the cervical seal 400. The elastomeric portion 406 is tapered, extending from an edge of the first portion 402 to an edge of the second portion 404. The elastomeric portion 406 further includes a plurality of ribs 407 arranged along a length of the elastomeric portion and protruding from a surface thereof. The ribs 407 are pliable so as to allow deflecting. As shown in FIG. 4A, a diameter of the ribs 407 changes along the length of the elastomeric portion 406, thereby forming tapered ribs. In other embodiments, the diameter of the ribs may be the same. In various embodiments, the thickness of the ribs may vary. In various embodiments, the spacing between the ribs may also vary. For example, the ribs may be spaced such that upon deflecting the neighboring ribs do not overlap. In other embodiments, the ribs may be spaced such that upon deflecting the neighboring ribs overlap at least partially. In yet other embodiments, the spacing between each pair of neighboring ribs may vary along a length of the elastomeric portion that includes the ribs.

FIG. 4B shows a cross sectional view of the cervical seal 400, further illustrating a rigid structure 408. The rigid structure 408 includes a tubular portion 408a extending between the first portion 402 and the second portion 404. The tubular portion 408a includes a hollow tube having a diameter 410 sized to receive an elongated member of a cervical or intrauterine device. The elongated member may be a sheath of an intrauterine device and the sheath may be inserted through the tubular portion 408a of the cervical seal 400, as shown for example in FIG. 10. The rigid structure 408 further includes a flange 408b protruding radially outward from one end of the tubular portion 408a. The first portion 402 includes the flange 408b. In some embodiments, the rigid structure may be made of a plastic material or a metal. As shown in FIG. 4B, the elastomeric portion 406 including the ribs 407 is attached to the rigid structure 408 and extends from the first portion 402 to the second portion 404 along a length of the tubular portion 408a.

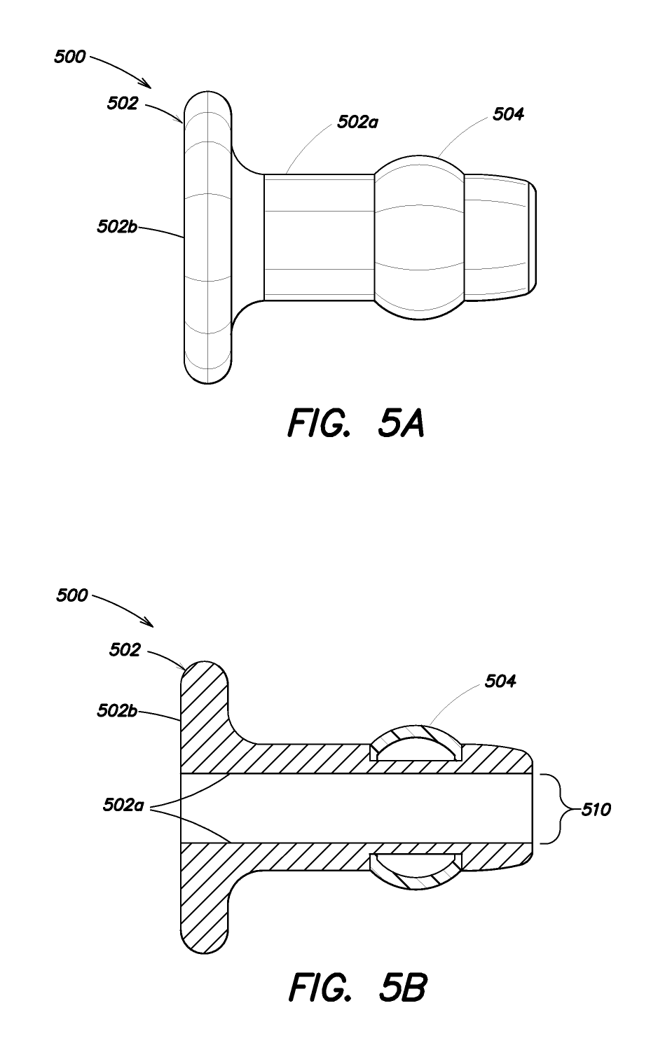

FIG. 5A illustrates another embodiment of a cervical seal 500 including a rigid structure 502 and an elastomeric portion 504. The rigid structure 502 includes a tubular portion 502a and a flange 502b protruding radially outward from a first end of the tubular portion. The flange 502b has a diameter larger than a diameter of the second end of the tubular portion 502a. The elastomeric portion 504 includes a localized bump disposed on the tubular portion 502a of the rigid structure 502. The localized bump may, for example, facilitate localized sealing of a specific portion of the cervical anatomy.

FIG. 5B is a cross sectional view of the cervical seal 500, showing the rigid structure 502 including the tubular portion 502a and the flange 502b, and the elastomeric portion 504 disposed on the tubular portion 502a as a localized bump. The tubular portion 502a includes a hollow tube having a diameter 510 sized to receive an elongated member of a cervical or intrauterine device. The elongated member may be a sheath of an intrauterine device and the sheath may be inserted through the tubular portion 502a of the cervical seal 500, as shown for example in FIG. 10. In some embodiments, the cervical seal may include an elastomeric portion having a plurality of localized bumps. In various embodiments, the length, width and height of localized bumps may vary. In FIGS. 5A and 5B, the elastomeric portion 504 is wrapped around a cylindrical area of the tubular portion 502a. In other embodiments, the elastomeric portion may be partially wrapped around the tubular portion. In some embodiments, the rigid structure may be made of a plastic material or a metal.

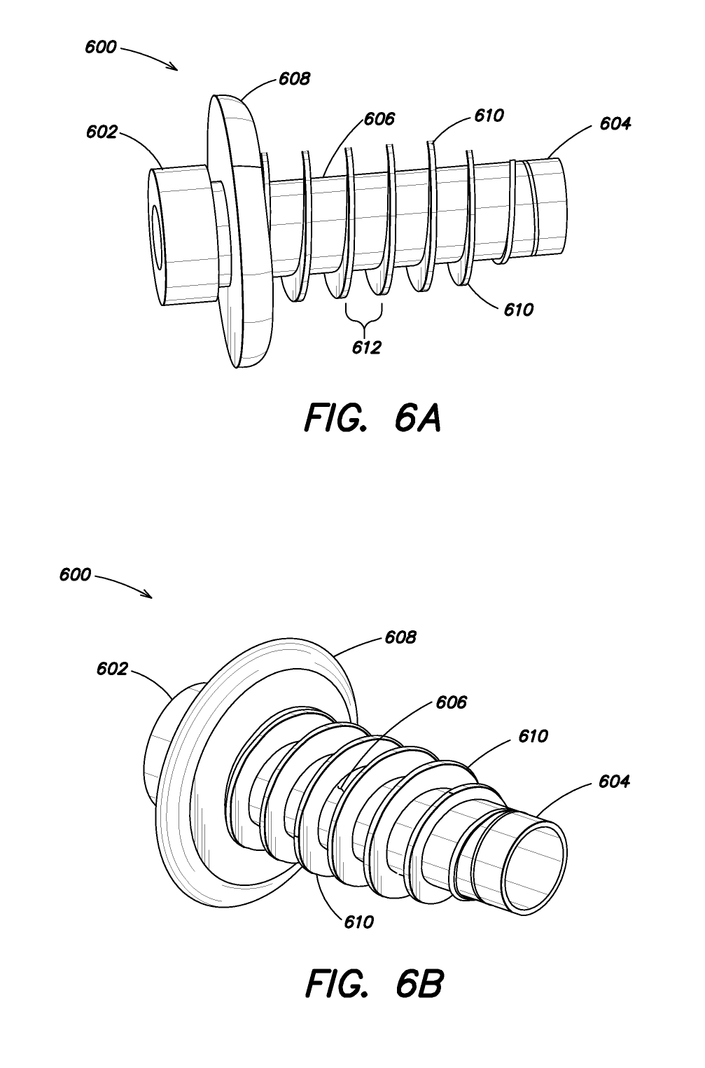

Referring to FIGS. 6A and 6B, there is illustrated another embodiment of a cervical seal 600 including a first portion 602, a second portion 604 and an elastomeric portion 606 coupled to the first portion and the second portion. In some embodiments, the entire cervical seal may be elastomeric. In other embodiments, the elastomeric portion may be coupled to a rigid structure, for example a structure made of a plastic or metal. The first portion 602 and the second portion 604 form respective ends of the cervical seal 600. The first portion 602 includes a flange 608 having a diameter larger than a diameter of the second portion 604 of the cervical seal 600. The elastomeric portion 606 is cylindrical, extending from the first portion 602 to the second portion 604. The elastomeric portion 606 further includes a helical rib 610 disposed around a length of the elastomeric portion. The helical rib 610 protrudes outward from a surface of the tubular elastomeric portion. The helical rib 610 is pliable so as to allow deflecting. As shown in FIGS. 6A and 6B, a diameter of the helical rib 610, or a diameter of a plurality of edges of the helical rib also changes along the length of the elastomeric portion 606, thereby forming tapered ribs or edges. In other embodiments, the diameter of the helical rib may be the same. In various embodiments, the thickness of the helical rib may vary. In various embodiments, a spacing 612 between neighboring ribs or edges of the helical rib may also vary. For example, neighboring ribs may be spaced such that upon deflecting the neighboring ribs do not overlap. In other embodiments, the neighboring ribs may be spaced such that upon deflecting the neighboring ribs overlap at least partially. In yet other embodiments, the spacing between each pair of neighboring ribs of the helical rib may vary along a length of the elastomeric portion.

Each of the first portion 602, the second portion 604 and the elastomeric portion 606 includes a hollow center having a diameter sized to receive an elongated member of a cervical or intrauterine device. The elongated member may be a sheath of an intrauterine device and the sheath may be inserted through the hollow center of the cervical seal 600, as shown for example in FIG. 10. In some embodiments, the cervical seal 600 may include a rigid structure having a tubular portion extending between the first portion 602 and the second portion 604 and the elastomeric portion 606 may be disposed on the tubular portion of the rigid structure. In other embodiments, only the first portion 602 and the second portion 604 may include a rigid structure and the elastomeric portion 606 may extend between the first portion and the second portion. In yet other embodiments, the cervical seal 600 may not include a rigid structure.

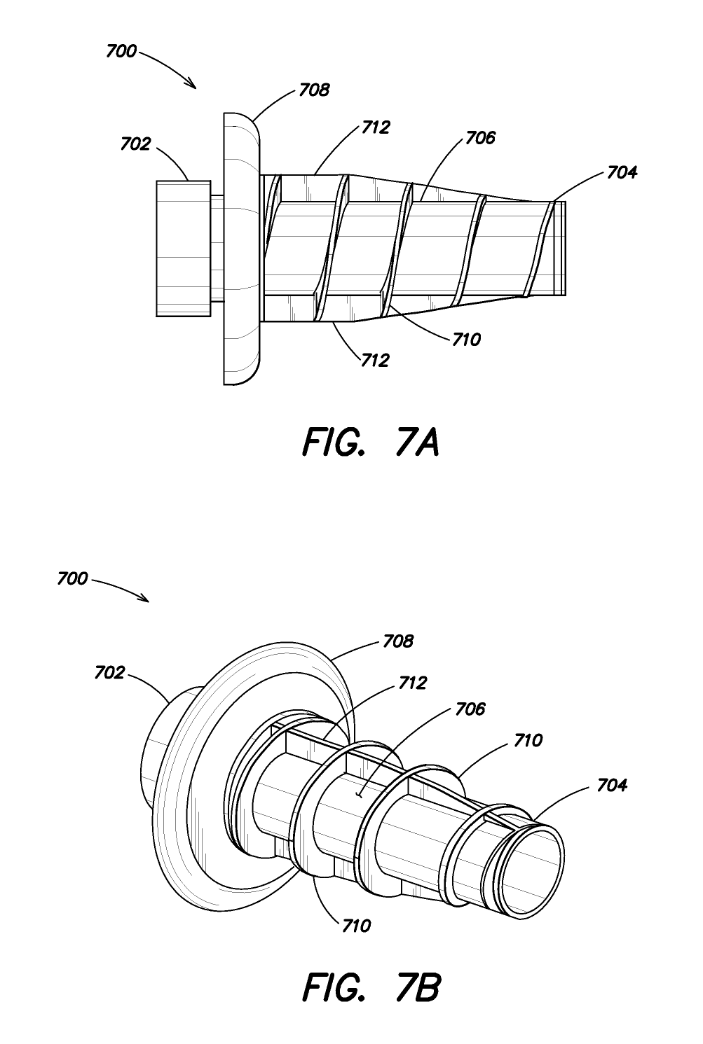

FIGS. 7A and 7B illustrate another embodiment of a cervical seal 700. The cervical seal 700 is configured similarly to the cervical seal 600 and includes a first portion 702, a second portion 704, and an elastomeric portion 706 coupled to the first portion and the second portion. In some embodiments, the entire cervical seal may be elastomeric. In other embodiments, the elastomeric portion may be coupled to a rigid structure, for example a structure made of a plastic or metal. The first portion 702 and the second portion 704 form respective ends of the cervical seal 700. The first portion 702 includes a flange 708 having a diameter larger than a diameter of the second portion 704 of the cervical seal 700. The elastomeric portion 706 is cylindrical, extending from the first portion 702 to the second portion 704. The elastomeric portion 706 further includes a helical rib 710 disposed around a length of the elastomeric portion and longitudinal ribs 712 disposed along the length of the elastomeric portion and coupled to the helical rib. For example, each longitudinal rib 712 is shown to be coupled to a plurality of edges of the helical rib 710. The helical rib 710 and the longitudinal ribs 712 protrude outward from a surface of the tubular elastomeric portion. The helical rib 710 and the longitudinal ribs 712 are pliable so as to allow deflecting. The longitudinal ribs 712 are tapered, having a variable height along the length of the elastomeric portion 706. In other embodiments, the longitudinal ribs may not be tapered. Various embodiments may include one or more longitudinal ribs having different lengths, thicknesses and heights. As shown in FIGS. 7A and 7B, a diameter of the helical rib 710, or a diameter of a plurality of edges of the helical rib also changes along the length of the elastomeric portion 706, thereby forming tapered ribs or edges. In other embodiments, the diameter of the helical rib may be the same. In various embodiments, the thickness of the helical rib may vary. In various embodiments, the spacing between neighboring ribs or edges of the helical rib may also vary. For example, neighboring ribs may be spaced such that upon deflecting the neighboring ribs do not overlap. In other embodiments, the neighboring ribs may be spaced such that upon deflecting the neighboring ribs overlap at least partially. In yet other embodiments, the spacing between a pair of neighboring ribs of the helical rib may vary along a length of the elastomeric portion.

Each of the first portion 702, the second portion 704 and the elastomeric portion 706 includes a hollow center having a diameter sized to receive an elongated member of a cervical or intrauterine device. The elongated member may be a sheath of an intrauterine device and the sheath may be inserted through the hollow center of the cervical seal 700, as shown for example in FIG. 10. In some embodiments, the cervical seal 700 may include a rigid structure having a tubular portion extending between the first portion 702 and the second portion 704 and the elastomeric portion 706 may be disposed on the tubular portion of the rigid structure. In other embodiments, only the first portion 702 and the second portion 704 may include a rigid structure and the elastomeric portion 706 may extend between the first portion and the second portion. In yet other embodiments, the cervical seal 700 may not include a rigid structure.

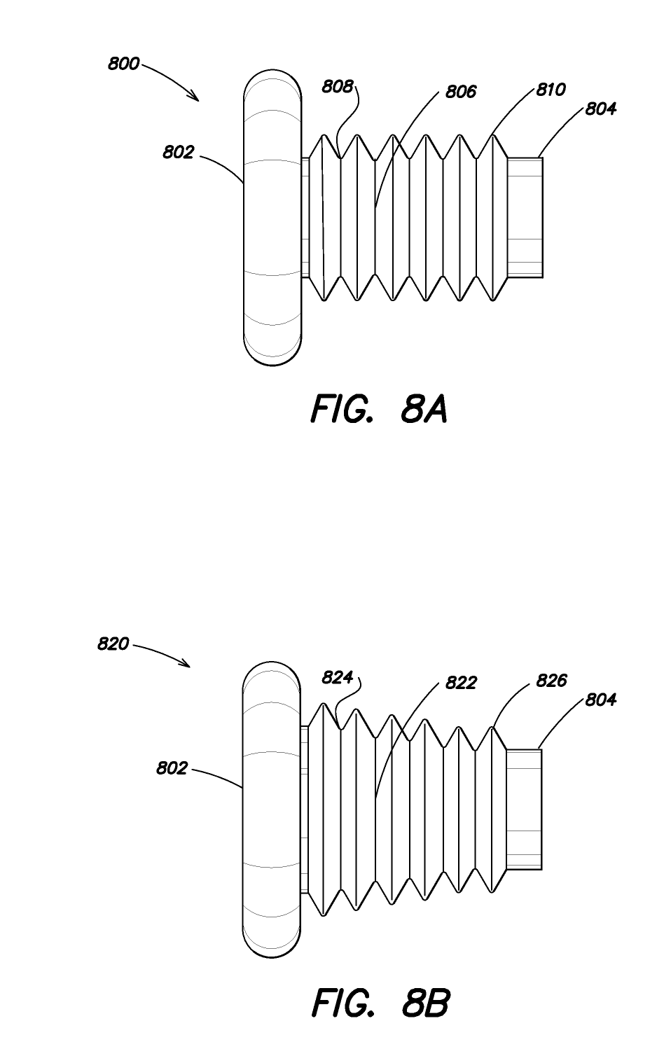

FIG. 8A illustrates another embodiment of a cervical seal 800 including a first portion 802, a second portion 804, and an elastomeric portion 806 coupled to the first portion and the second portion. In some embodiments, the entire cervical seal may be elastomeric. In other embodiments, the elastomeric portion may be coupled to a rigid structure, for example a structure made of a plastic or metal. In yet other embodiments, the cervical seal may not include an elastomeric portion and may include a tubular portion made of another material. The first portion 802 and the second portion 804 form respective ends of the cervical seal 800. The first portion 802 includes a flange having a diameter larger than a diameter of the second portion 804 of the cervical seal 800. The elastomeric portion 806 has an accordion shape including a plurality of alternating inner rims 808 and outer rims 810 along a length of the elastomeric portion. The elastomeric portion 806 extends from the first portion 802 to the second portion 804. The accordion shape allows at least a portion of the cervical seal to be flexible, thereby expanding or contracting.

Each of the first portion 802, the second portion 804 and the elastomeric portion 806 includes a hollow center having a diameter sized to receive an elongated member of a cervical or intrauterine device. The elongated member may be a sheath of an intrauterine device and the sheath may be inserted through the hollow center of the cervical seal 800, as shown for example in FIG. 10. In some embodiments, the cervical seal 800 may include a rigid structure having a tubular portion extending between the first portion 802 and the second portion 804 and the elastomeric portion 806 may be disposed on the tubular portion of the rigid structure. In other embodiments, only the first portion 802 and the second portion 804 may include a rigid structure and the elastomeric portion 806 may extend between the first portion and the second portion. In yet other embodiments, the cervical seal 800 may not include a rigid structure.

The accordion shape may further be tapered, as shown for example in FIG. 8B. The cervical seal 820 includes an elastomeric portion 822 having an accordion shape including alternative inner rims 824 and outer rims 826. A diameter of each of the inner rims 824 and the outer rims 826 varies along the length of the elastomeric portion 822.

As described above in relation with various embodiments of the cervical seals disclosed in FIGS. 1 to 8, a cervical seal may include a rigid structure having a tubular portion extending between a first portion and a second portion of the cervical seal, and may further include an elastomeric portion or a membrane covering at least a portion of the rigid structure. In other embodiments, only the first portion and the second portion may include a rigid structure and an elastomeric portion or a membrane may be coupled to at least one of the first portion and the second portion. The elastomeric portion may include one or more features described in relation with the embodiments of FIGS. 1 to 8. In these embodiments, the elastomeric portion or membrane may be flexible so as to allow the length of the cervical seal to be adjusted, for example as described and illustrated further below. In yet other embodiments, a cervical seal may not include a rigid structure. For example, the entire cervical seal may be made of an elastomeric material or another flexible material.

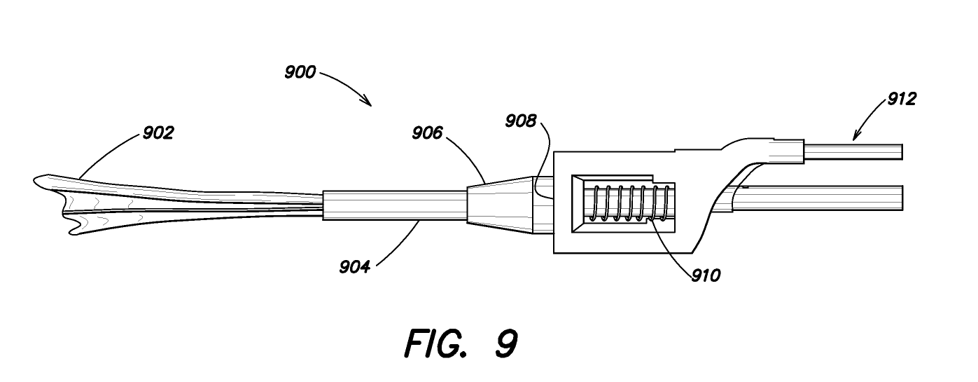

FIG. 9 illustrates an intrauterine therapy application device 900 including an applicator 902, a sheath 904, and a cervical seal 906. As shown in FIG. 9, the applicator 902 is in a deployed position. According to one embodiment, the applicator 902 may be retracted in a collapsed position within the sheath 904. The sheath 904 may be inserted through the patient's cervix, and the applicator 902 of the intrauterine therapy application device 900 may be extended out from the sheath 904 in a collapsed position and may be expanded in the uterus to allow therapy application. The sheath 904 is an elongated central member that extends through a hollow tube of the cervical seal 906. In some embodiments, the cervical seal may be coupled to the sheath. In some embodiments, the cervical seal may be configured to allow at least a portion of the sheath to slide through the cervical seal. In some embodiments, to be discussed herein, the position of the cervical seal along the sheath may be adjusted for example based on the length of the cervical canal of a patient.

In various embodiments, the cervical seal 906 may be any one of the cervical seals disclosed herein. For example, the cervical seal 906 may include one or more features described in relation with the embodiments of FIGS. 1 to 8. The cervical seal 906 includes a flange 908 and the flange is loaded by a spring 910. The flange is further coupled to a proximal portion 912 of the intrauterine device 900. The flange may be configured to have a diameter larger than a diameter of the cervical anatomy, such as the external os of the cervical anatomy and may be configured to provide a physical reference from the cervical seal to the cervical anatomy. In various embodiments, the cervical seal may be coupled to an elongated central member such as the sheath 904 of FIG. 9 and the elongated central member may be coupled to at least one of a gynecological surgical device, an ablation device, a cervical device and a uterine device. In these embodiments, the cervical seal may be disposed along the sheath and configured so as to seal at least one of the external os, a portion of the cervical canal and the internal os of the cervix.

FIG. 10 illustrates an intrauterine therapy application device 1000 including a sheath 1002, a cervical seal 1004 and a proximal portion 1006 coupled to the cervical seal. For example, the proximal portion 1006 may be coupled to a flange 1007 of the cervical seal 1004. In various embodiments, the cervical seal 1004 may be any one of the cervical seals disclosed herein. For example, the cervical seal 1004 may include one or more features described in relation with the embodiments of FIGS. 1 to 8. The sheath 1002 may be calibrated and marked with a scale, for example in a centimeter scale. The sheath 1002 is an elongated central member that extends through a hollow tube of the cervical seal 1004. In some embodiments, the cervical seal 1004 may be coupled to the sheath. In some embodiments, the cervical seal 1004 may be configured to allow at least a portion of the sheath to slide through the cervical seal. The proximal portion 1006 includes buttons 1008 to allow or otherwise lock movement of the sheath 1002 so as to set the position of the cervical seal 1004. For example, the position of the seal 1004 relative to the sheath 1002 may be set and locked. In some embodiments, the length of the sheath 1002 may be adjusted by sliding the sheath relative to the cervical seal 1004. In some embodiments, the cervical seal 1004 may be an adjustable length cervical seal. For example as described and illustrated further below, the length of the cervical seal 1004 may be adjusted along the sheath 1002 by extending one end of the cervical seal along the sheath. The length of the cervical seal along the sheath may be adjusted for example based on the length of the cervical canal of a patient. The buttons 1008 may be configured for ambidextrous use. The proximal portion 1006 further includes an indicator 1010 configured to display a position of the sheath 1002 or the cervical seal 1004. For example, the indicator 1010 may include a port or window to allow viewing of a marking on the sheath. For example, the marking may be indicative of the length of the sheath or the length of the cervical seal.

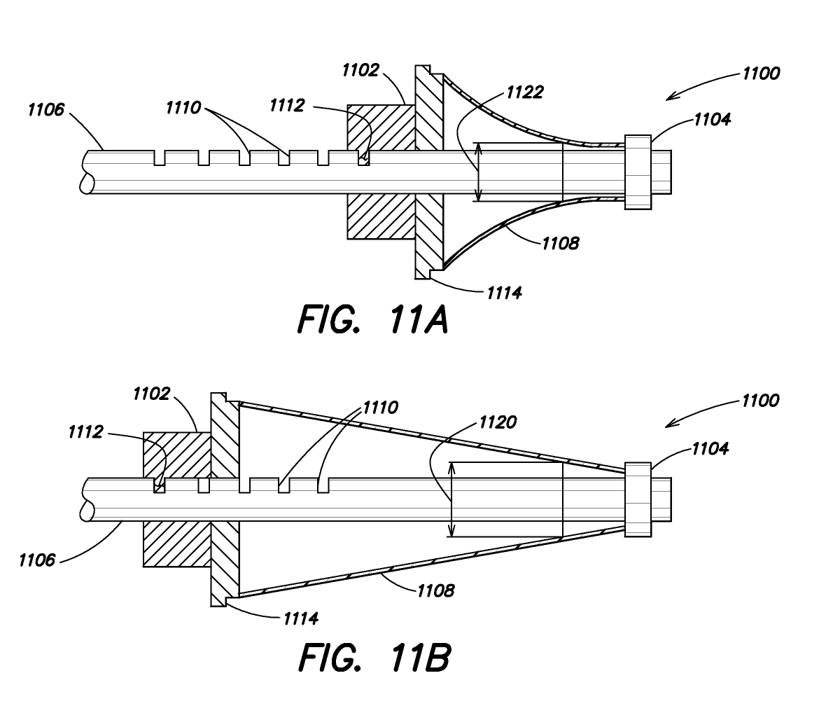

FIG. 11A illustrates an exemplary embodiment of a cervical seal 1100 having an adjustable length according to aspects of the present disclosure. The adjustable length cervical seal 1100 includes a first portion 1102 and a second portion 1104, each disposed around an elongated central member 1106. Each of the first portion 1102 and the second portion 1104 includes a hollow central portion configured to receive the elongated central member 1106.

The length of the cervical seal may correspond to a distance between the first portion 1102 and the second portion 1104 along the elongated central member 1106. In this embodiment, the second portion 1104 is coupled to the elongated central member 1106, whereas the first portion 1102 is movable along the elongated central member 1106. In other embodiments of the adjustable length cervical seal, the first portion 1102 may be coupled to the elongated central support member 1106 and the second portion 1104 may be movable along the elongated central support member. In yet other embodiments, both the first portion 1102 and the second portion 1104 may be movable along the elongated central member 1106. In various embodiments, at least one of the first portion and the second portion of an adjustable length cervical seal may be movable from a first position to a second position along the elongated central member, so as to change the length of the cervical seal.

The elongated central member 1106 may include a sheath, for example as illustrated in FIGS. 9 and 10. In some embodiments, the elongated central member may further include a proximal portion as illustrated for example in FIG. 10. In some embodiments, the elongated central member may include telescoping portions, such as a first cylindrical portion and a second cylindrical portion configured to slide through the first cylindrical portion. In one example, the first portion of the adjustable cervical seal may be coupled to the first cylindrical portion and the second portion of the adjustable cervical seal may be coupled to the second cylindrical portion of the telescoping portions. As the second cylindrical portion extends through the first cylindrical portion, the length of the cervical seal is increased. The first cylindrical portion may be a proximal portion or a handle of an intrauterine device and the second cylindrical portion may be a sheath.

The cervical seal 1100 further includes a membrane 1108 coupled to at least one of the first portion 1102 and the second portion 1104. In this embodiment, the membrane 1108 is coupled to the first portion 1102 and the second portion 1104, wherein each of the first portion and the second portion includes a rigid structure. In other embodiments, the membrane may be coupled, for example, to the first portion that is movable along the elongated central member and may further be coupled directly to at least a portion of the elongated central member forming a second portion of the cervical seal, wherein the second portion may not include a rigid structure. The membrane 1108 is configured to stretch and contract in response to increasing and decreasing the length of the cervical seal 1100. In some embodiments, the membrane 1108 may be a conformable membrane. For example, the membrane 1108 may conform to the shape of the cervical anatomy. In some embodiments, the membrane 1108 may include a variable durometer material along a length of the cervical seal. A variable durometer may enable the membrane 1108 to take an irregular form at a desired location of the cervical seal, for example to conform to an irregular shape of a cervical anatomy. In one example, a distal end of the membrane 1108 such as a portion of the membrane close to the second portion 1104 may be more pliable relative to a proximal end of the membrane, thereby allowing the distal end of the membrane to deform more than the proximal end. A variable durometer membrane may allow non-uniform expansion of the membrane and sealing of a non-uniform anatomy. In some embodiments, the membrane 1108 may be a sealed balloon membrane that includes a fluid. The fluid may support the membrane 1108 and allow it to conform.

In some embodiments, the cervical seal may include the elongated central member. In other embodiments, the elongated central member may be associated with an intrauterine device. In various embodiments, a cervical seal may be configured to lock at least one of the first portion and the second portion that is movable to the elongated central member. For example, the cervical seal 1100 is configured to lock the first portion 1102 that is movable to the elongated central member 1106. For example, the elongated central member 1106 includes a plurality of grooves 1110 arranged along the elongated central member and being configured to receive a locking portion 1112 of the first portion 1102. In other embodiments, the second portion 1104 may be movable along the elongated central member 1106 and the second portion may include a locking portion so as to allow locking the second portion to the elongated central member. In various embodiments, the plurality of grooves may be arranged to provide a plurality of predetermined lengths of the cervical seal. For example, the distance between pairs of neighboring grooves 1110 or the number of grooves may be configured according to a plurality of predetermined lengths desirable for the cervical seal 1100.

FIG. 11B illustrates the adjustable length cervical seal of FIG. 11A having an increased length according to aspects of the present invention. As shown, the second portion 1104 is affixed to the elongated central member 1106, whereas the first portion 1102 has been moved and the locking portion 1112 has been coupled to a different groove 1110 of the plurality of grooves of the elongated central member. The membrane 1108 is tapered and is shown to be stretched in response to increasing the length of the cervical seal. In some embodiments, the tension in the stretched membrane may allow the membrane to conform to the cervical anatomy, thereby sealing the cervical anatomy. For example, the membrane 1108 may be configured to seal at least a portion of a cervical canal.

The cervical seal 1100 may be configured to seal at least one of the external os and the internal os of a cervix. The first portion 1102 includes a flange 1114 having a diameter larger than a diameter of the second portion 1104. The membrane 1108 extends from an outer edge of the flange 1114 to an outer edge of the second portion 1104. The membrane 1108 is tapered such that a diameter of the membrane changes along at least a portion of the cervical seal 1100. As shown in FIG. 11B, as the length of the cervical seal 1100 is increased, the membrane 1108 is stretched and a radial profile of the membrane increases. An increase in the radial profile of the membrane 1108 may correspond to an increase in a diameter of the membrane at one or more locations along the cervical seal. Referring to FIGS. 11A and 11B, as the first portion 1102 moves away from the second portion 1104 to increase the length of the cervical seal, the diameter 1120 of the membrane 1108 at a location along the length of the cervical seal may increase relative to a corresponding diameter 1122 of the membrane at a same distance from the second portion 1104 when the cervical seal length is not increased. When the cervical seal having a nominal length as shown in FIG. 11A is extended as shown in FIG. 11B, tension in the membrane 1108 displaces the membrane outward. At any given distance from an end portion of the cervical seal, the diameter of the membrane 1108 is greater when the cervical seal is extended. As described above, the membrane 1108 may have a variable durometer, for example to provide non-uniform sealing. A variable durometer membrane may allow non-uniform expansion of the membrane as the length of the cervical seal is increased, thereby sealing a non-uniform anatomy.

In various embodiments, a diameter of the proximal end of the membrane 1100 coupled to the flange 1114 may remain unchanged as the length of the cervical seal is increased. A diameter of the distal end of the membrane 1100 coupled to the second portion 1104 may also remain unchanged as the length of the cervical seal is increased.

The flange 1114 may provide a physical reference from the cervical seal 1100 to a cervical anatomy. The diameter of the flange 1114 may be larger than a diameter of a cervical anatomy. In one example, the length of the cervical seal 1100 may be adjusted such that the first portion 1102 including the flange 1114 may be located at the external os of the cervix, the second portion 1104 may be located at the internal os of the cervix, and the membrane 1108 may be stretched to seal the cervical canal. In other embodiments, the first portion may include a pliable flange and the cervical seal may be disposed along an elongated central member such that the pliable flange may be inserted through the cervical canal and may be located so as to seal the internal os of the cervix, whereas the second portion having a smaller diameter than the flange may be located at the external os of the cervix.

In various embodiments, the adjustable length cervical seal 1100 may include one or more features of any one of the cervical seals disclosed herein. For example, the cervical seal 1100 may include one or more features described in relation with the embodiments of FIGS. 1 to 8. For example, the membrane 1108 may be made of an elastic material. The first portion 1102 and the second portion 1104 may include a rigid structure made of one of a plastic material and a metal. In other embodiments, the first portion 1102, the second portion 1104 and the membrane 1108 may each be made of an elastomeric material. A rigid substructure may be disposed beneath at least one of the first portion and the second portion.

In various embodiments, the first portion 1102 and the second portion 1104 may be the first and second portions forming respective ends of a cervical seal as described herein, such as in the embodiments described and illustrated in FIGS. 1 to 8. In an adjustable length cervical seal, the first and second portions may not be coupled by a rigid tubular portion. The membrane 1108 may include one or more features of the elastomeric portions described and illustrated for example in the embodiments of FIGS. 1 to 8. In one example, the membrane 1108 may include a webbing, a thin membrane supported by elasticity or a self-supporting membrane as illustrated for example by membrane 106 in FIGS. 1A and 1B. The membrane 1108 may include an inflatable balloon or a sealed balloon including a fluid providing support to the membrane, as illustrated for example by the membrane 206 in FIGS. 2A and 2B. The membrane 1108 may include any flexible material and may be configured as a dilator to facilitate insertion into the cervical canal.

In some embodiments, the membrane 1108 may have a tubular shape. The membrane 1108 may be cylindrical or tapered. An adjustable length cervical seal such as the seal 1100 may further include a plurality of ribs disposed along a length of the membrane and protruding radially from a surface of the membrane as shown for example in FIGS. 4A and 4B. The plurality of ribs may be pliable so as to allow deflecting. In some embodiments having a membrane and ribs disposed thereon, as the membrane stretches the spacing between the ribs may change. In some embodiments, a diameter of the ribs may change along the length of the membrane.

In some embodiments, the membrane 1108 may have a tubular shape and the cervical seal may further include a helical rib disposed around the membrane and along a length of the membrane, as shown for example in FIGS. 6 and 7. The cervical seal may further include one or more longitudinal ribs disposed along the length of the membrane and coupled to a plurality of edges of the helical rib as shown for example in FIGS. 7A and 7B. One or more of the helical rib and the longitudinal ribs may be tapered. For example, a diameter of a plurality of edges of the helical rib may change along the length of the membrane and a longitudinal rib may be tapered.

In yet other embodiments, the membrane 1108 may have an accordion shape including a plurality of alternating inner and outer annular rims as shown for example in FIGS. 8A and 8B. The accordion shaped membrane may be tapered along at least a portion of the cervical seal. For example, a respective diameter of each annular rim of the plurality of alternating inner and outer annular rims may change along a portion of the length of the cervical seal. The accordion shape of the membrane 1108 allows the membrane to stretch and contract as the length of the cervical seal increases and decreases, respectively.

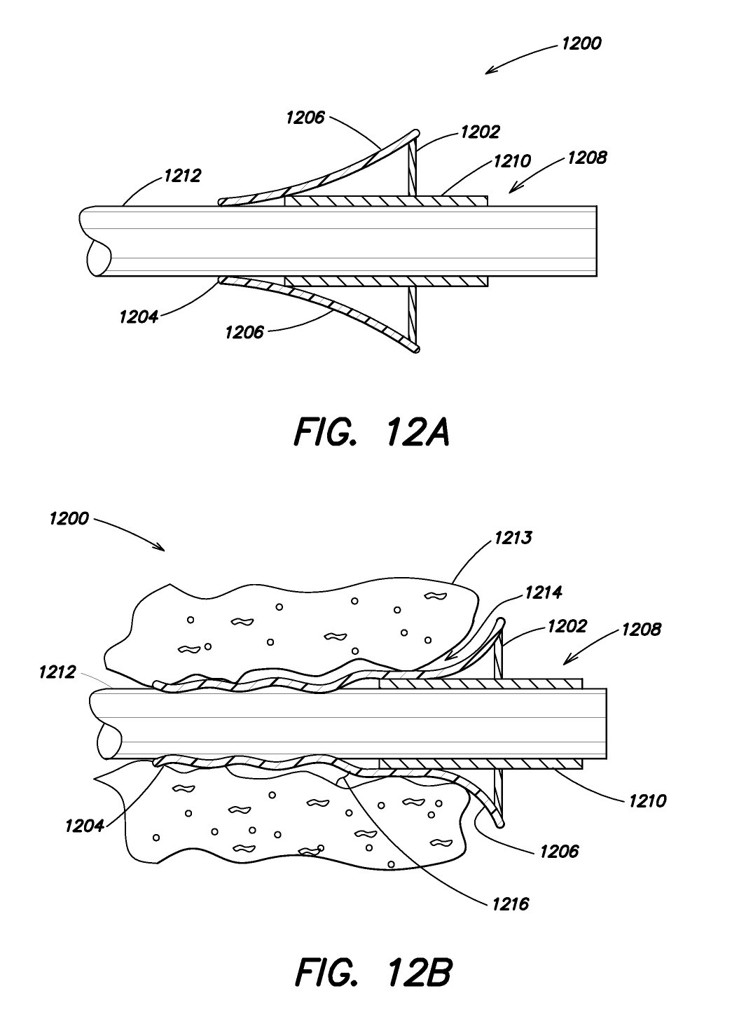

FIG. 12A illustrates another embodiment of a cervical seal 1200 having an adjustable length. The cervical seal 1200 includes a first portion 1202 having a diameter larger than a second portion 1204 of the cervical seal. The cervical seal further includes a membrane 1206 that is tapered and extending from the first portion 1202 to the second portion 1204. The cervical seal further includes an elongated central member 1208. The elongated central member 1208 has a telescoping configuration and includes a first cylindrical portion 1210 coupled to the first portion 1202 of the cervical seal 1200, and a second cylindrical portion 1212 that may slide relative to the first cylindrical portion. The second cylindrical portion 1212 is bonded to the membrane 1206 to form the second portion 1204 of the cervical seal. The second cylindrical portion 1212 may be a sheath and the first cylindrical portion 1210 may be coupled to a handle or proximal portion of an intrauterine device. As the first cylindrical portion 1210 is fixed and the second cylindrical portion 1212 is pushed forward relative to the first cylindrical portion, the length of the cervical seal increases and the membrane 1206 stretches.

FIG. 12B shows the cervical seal 1200 having an increased length and sealing a cervical anatomy 1213 including the external os 1214 and a portion of the cervical canal 1216. The cervical anatomy 1213 may have an unusual shape. The membrane 1206 is flexible and is shown to conform to the shape of the cervical canal 1216 to seal the cervical canal. The first portion 1202 of the cervical seal 1200 includes a flange having a diameter larger than that of the external os 1214 of the cervix, thereby sealing the external os and providing a physical reference or locating flange. The cervical seal 1200 may include one or more features disclosed herein in relation with various embodiments of cervical seals.

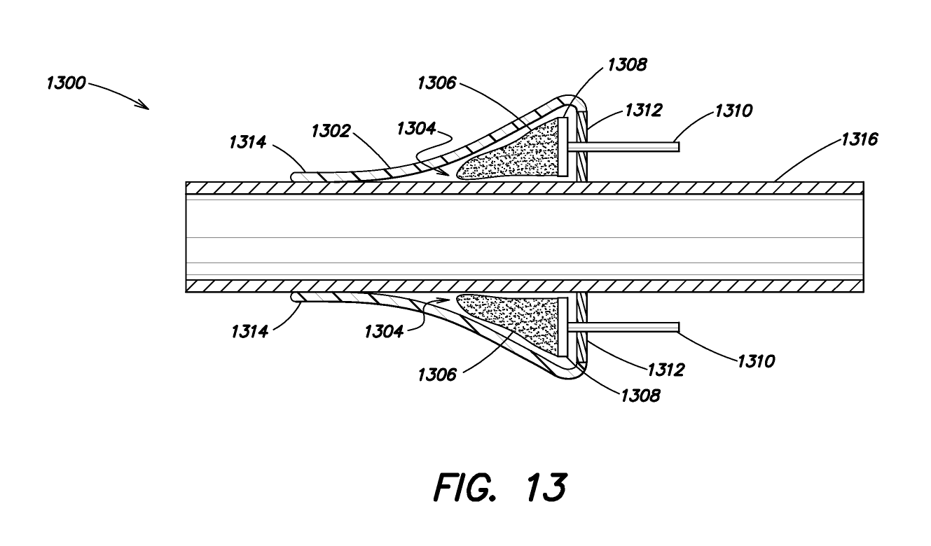

FIG. 13 illustrates an exemplary embodiment of a cervical seal 1300 having a membrane 1302 backed by at least one conformable structure 1304. The conformable structure 1304 includes a conformable foam 1306 and a backing plate 1308. The backing plate 1308 may not be conformable. As shown in FIG. 13, the conformable structure 1304 and more specifically the backing plate 1308 is coupled to a push rod 1310 configured to adjust the conformable structure and to provide support to the membrane 1302. In other embodiments, the membrane 1302 may be backed by a spring-loaded structure or a spring-loaded foam. The cervical seal 1300 further includes a first portion 1312 including retainers configured to allow a push rod 1310 to slide through while maintaining the conformable structure 1304 localized behind the membrane 1302. The cervical seal 1300 further includes a second portion 1314 formed by bonding one end of the membrane 1302 to an elongated central member 1316 of the cervical seal. In some embodiments, the first portion 1312 may be coupled to the elongated central member 1316, thereby providing a cervical seal having a fixed length. In other embodiments, the first portion may be movable and lockable along the elongated central member 1316, thereby providing a cervical seal having an adjustable length.

In various embodiments, the cervical seal 1300 may be configured to include one or more features disclosed herein in relation with various embodiments of cervical seals. For example, the membrane 1302 may further include a plurality of ribs or may have an accordion shape as illustrated in FIGS. 6 and 8. The cervical seal 1300 may include a helical rib disposed around the membrane and along a length of the membrane. The cervical seal 1300 may further include one or more longitudinal ribs disposed along the length of the membrane and coupled to a plurality of edges of the helical rib as shown for example in FIGS. 7A and 7B. One or more of the helical rib and the longitudinal ribs may be tapered. For example, a diameter of a plurality of edges of the helical rib may change along the length of the membrane and a longitudinal rib may be tapered. In other embodiments, the membrane 1302 may have an accordion shape including a plurality of alternating inner and outer annular rims as shown for example in FIGS. 8A and 8B. The accordion shaped membrane may be tapered along at least a portion of the cervical seal. For example, a respective diameter of each annular rim of the plurality of alternating inner and outer annular rims may change along a portion of the length of the cervical seal. In other embodiments, the membrane 1302 may include a webbing, a thin membrane, a self-supporting membrane, an inflatable balloon or a sealed balloon. The membrane 1302 may include any flexible material and may be configured as a dilator to facilitate insertion into the cervical canal.

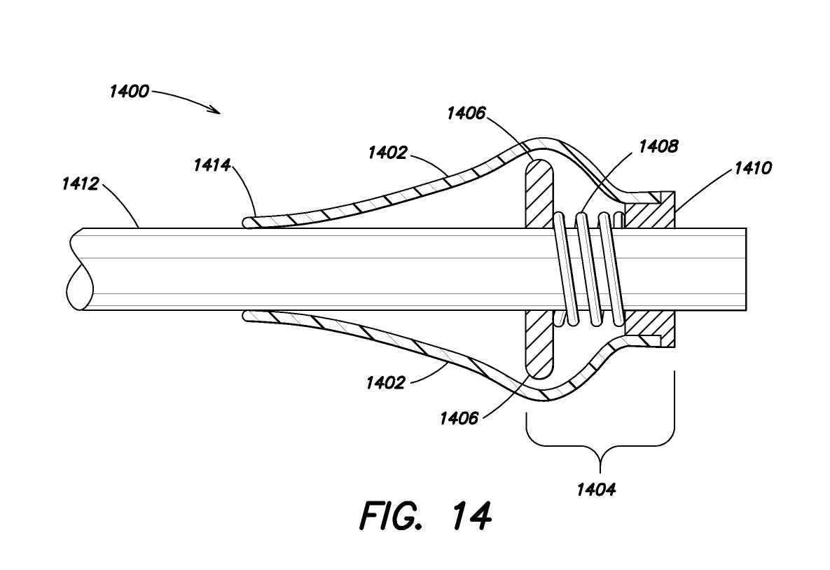

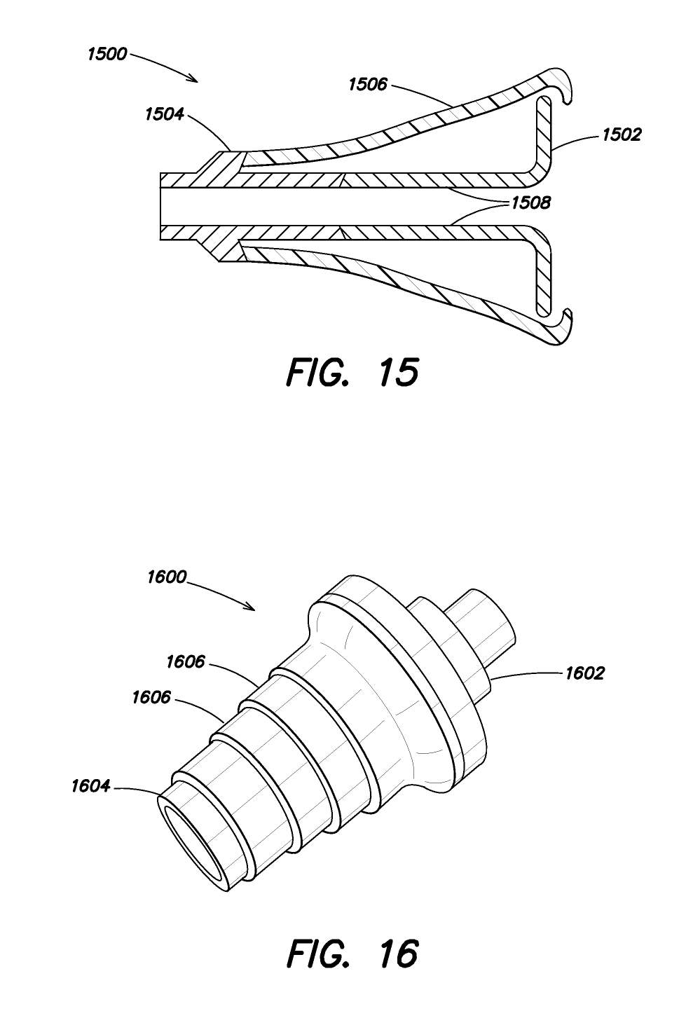

FIG. 14 illustrates an exemplary embodiment of a cervical seal 1400 having a membrane 1402 backed by at least one spring-loaded structure 1404. The spring-loaded structure may include a spring-loaded foam. The foam may be a deformable foam ring. The spring-loaded structure 1404 includes a first portion 1406 of the cervical seal 1400, a spring 1408 and an anchor 1410. For example, the first portion may be a flange and the flange may be spring-loaded. The spring 1408 is disposed between the first portion 1406 and the anchor 1410. In this embodiment, the membrane 1402 is not attached to the first portion 1406 and instead the membrane is pulled over the first portion and attached to the anchor 1410 that is coupled to the spring 1408. The anchor 1410 may be pulled back by the spring 1408 to maintain tension in the membrane 1402. The other end of the membrane 1402 is bonded to an elongated central member 1412 of the cervical seal 1400, thereby forming a second portion 1414 of the cervical seal. In some embodiments, the first portion 1406 may be coupled to the elongated central member 1412, thereby providing a cervical seal having a fixed length. In other embodiments, the first portion 1406 may be movable and lockable along the elongated central member 1412, thereby providing a cervical seal having an adjustable length.

In various embodiments, the cervical seal 1400 may be configured to include one or more features disclosed herein in relation with various embodiments of cervical seals. For example, the membrane 1402 may further include a plurality of ribs or may have an accordion shape as illustrated in FIGS. 6 to 8. The cervical seal 1400 may include a helical rib disposed around the membrane and along a length of the membrane. The cervical seal 1400 may further include one or more longitudinal ribs disposed along the length of the membrane and coupled to a plurality of edges of the helical rib as shown for example in FIGS. 7A and 7B. One or more of the helical rib and the longitudinal ribs may be tapered. For example, a diameter of a plurality of edges of the helical rib may change along the length of the membrane and a longitudinal rib may be tapered. In other embodiments, the membrane 1402 may have an accordion shape including a plurality of alternating inner and outer annular rims as shown for example in FIGS. 8A and 8B. The accordion shaped membrane may be tapered along at least a portion of the cervical seal. For example, a respective diameter of each annular rim of the plurality of alternating inner and outer annular rims may change along a portion of the length of the cervical seal. In other embodiments, the membrane 1402 may include a webbing, a thin membrane, a self-supporting membrane, an inflatable balloon or a sealed balloon. The membrane 1402 may include any flexible material and may be configured as a dilator to facilitate insertion into the cervical canal.

FIG. 15 illustrates an exemplary embodiment of a cervical seal 1500 having a first portion 1502, a second portion 1504, and a membrane 1506 coupled to the first portion and the second portion. The first portion 1502 includes a flange having a diameter larger than that of the second portion 1504 and the membrane 1506 is tapered, extending from an edge of the first portion to an edge of the second portion. In some embodiments, the first portion 1502 and second portion 1504 may be coupled by a tubular portion 1508 having a fixed length so as to provide a cervical 1500 seal having a fixed length, as shown for example in FIG. 15. In other embodiments, the first portion 1502 and the second portion 1504 may include respective portions of an elongated central member, the respective portions being arranged in a telescoping configuration so as to allow adjusting the length of the cervical seal 1500 by sliding the portions of the telescoping configuration relative to each other. The second portion 1504 of the cervical seal 1500 includes a contoured tip as shown for example in FIG. 15. The contoured tip may be configured to locate an internal os of a cervix. For example, the contoured tip may include a step as shown in FIG. 15. The first portion 1502 may have a diameter larger than a diameter of the external os of the cervix so as to locate the external os. The cervical seal 1500 may thus be configured to allow obtaining a measurement of the length of the cervical canal.

In various embodiments, the cervical seal 1500 may be configured to include one or more features disclosed herein in relation with various embodiments of cervical seals. For example, the membrane 1506 may include a plurality of ribs or may have an accordion shape as illustrated in FIGS. 6 to 8. The cervical seal 1500 may include a helical rib disposed around the membrane and along a length of the membrane. The cervical seal 1500 may further include one or more longitudinal ribs disposed along the length of the membrane and coupled to a plurality of edges of the helical rib as shown for example in FIGS. 7A and 7B. One or more of the helical rib and the longitudinal ribs may be tapered. For example, a diameter of a plurality of edges of the helical rib may change along the length of the membrane and a longitudinal rib may be tapered. In other embodiments, the membrane 1506 may have an accordion shape including a plurality of alternating inner and outer annular rims as shown for example in FIGS. 8A and 8B. The accordion shaped membrane may be tapered along at least a portion of the cervical seal. For example, a respective diameter of each annular rim of the plurality of alternating inner and outer annular rims may change along a portion of the length of the cervical seal. In other embodiments, the membrane 1506 may include a webbing, a thin membrane, a self-supporting membrane, an inflatable balloon or a sealed balloon. The membrane 1506 may include any flexible material and may be configured as a dilator to facilitate insertion into the cervical canal.

FIG. 16 illustrates another embodiment of a cervical seal 1600 having a step profile. The cervical seal 1600 includes a first portion 1602, a second portion 1604, and an elastomeric portion including a plurality of steps 1606. In some embodiments, the entire cervical seal may be elastomeric. In other embodiments, the elastomeric portion may be coupled to a rigid structure, for example a structure made of a plastic or metal. In yet other embodiments, the cervical seal may not include an elastomeric portion and may include a tubular portion made of another material. The first portion 1602 and the second portion 1604 form respective ends of the cervical seal 1600. The cervical seal 1600 has a hollow center with a diameter sized to receive an elongated member of a cervical or intrauterine device. The first portion 1602 includes a flange having a diameter larger than a diameter of the second portion 1604. In various embodiments, the cervical seal 1600 may be configured to include any number of steps 1606. The step profile is associated with an increased surface pressure at the step points.

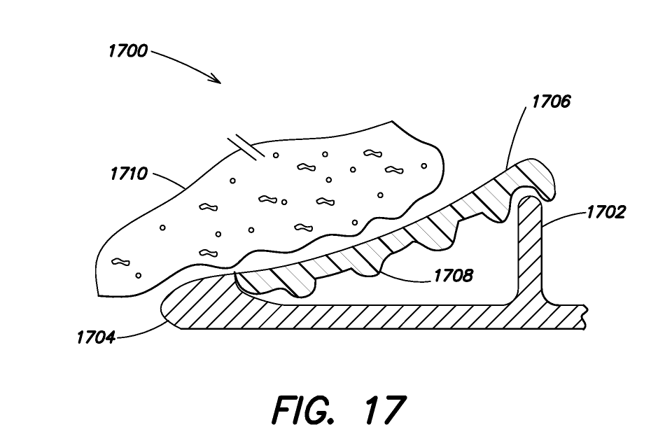

FIG. 17 illustrates a partial profile of another embodiment of a cervical seal 1700. The cervical seal 1700 includes a first portion 1702, a second portion 1704, and an elastomeric portion 1706 extending between the first and second portions. The elastomeric portion may be a membrane as described with reference to various embodiments. The elastomeric portion 1706 has a smooth exterior profile and a ribbed interior profile. A plurality of ribs 1708 are disposed on the interior of the elastomeric portion 1706. In various embodiments, the cervical seal 1700 may be configured to include any number of interior ribs 1708. The internal ribs 1708 may create variable firmness in the volute profile of the cervical seal. This allows the exterior of the elastomeric portion 1706 to be in contact with cervical tissue 1710 which may be non-uniform while the variable stiffness creates pressure concentrations for better sealing.

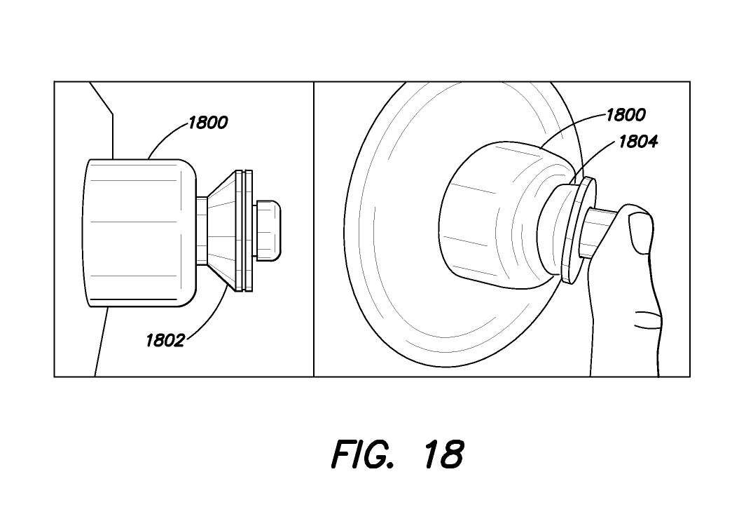

FIG. 18 illustrates an exemplary embodiment of a cervical seal inserted into a cervical anatomy 1800. The cervical seal has a first profile 1802 in a first position and a second profile 1804 in a second position responsive to the application of forward pressure on the seal to create the second profile. The expansion of the cervical seal may be non-uniform and may be used to seal a non-uniform cervical anatomy.

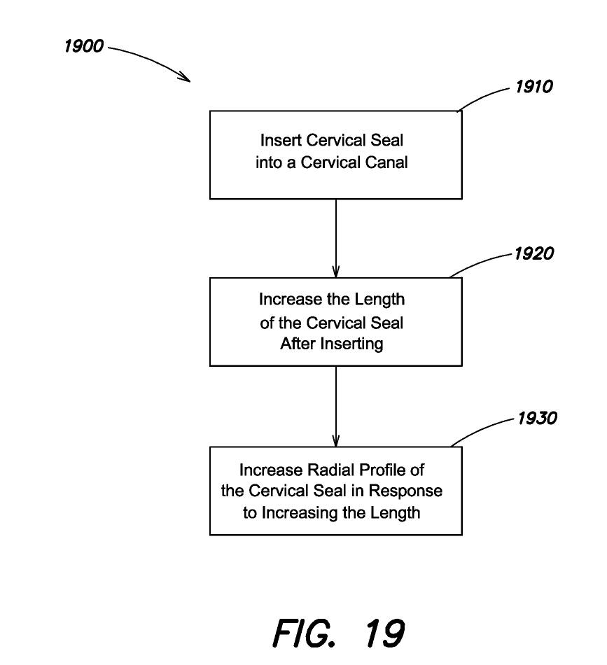

Another aspect of the present disclosure is directed to providing methods of sealing a cervical anatomy. FIG. 19 illustrates a flow chart of an exemplary method 1900 of sealing a cervical canal. The method includes an act 1910 of inserting a cervical seal into a cervical canal. The cervical seal may be an adjustable length cervical seal as described herein with reference to various embodiments. The method includes an act 1920 of increasing the length of the cervical seal after insertion into the cervical canal. The length of the cervical seal can be increased according to the various embodiments described herein. In other embodiments, the length of the cervical seal may be increased during insertion or the act of increasing the length may overlap with insertion into the cervical canal. The length of the cervical seal may be increased, for example, by moving a first end of the cervical seal relative to the second end of the cervical seal along a central sheath. The method further includes an act 1930 of increasing a radial profile of the cervical seal in response to increasing the length of the cervical seal. For example, as the length of the seal is increased, tension in the membrane displaces the membrane outward, thereby increasing the radial profile of the seal. Increasing the radial profile of the cervical seal may correspond to increasing a diameter of a membrane of the cervical seal at one or more locations along the length of the cervical seal. Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the disclosure. Accordingly, the foregoing description and drawings are by way of example only, and the scope of the disclosure should be determined from proper construction of the appended claims, and their equivalents.

What is claimed is:

1. A cervical seal having an adjustable length, comprising:
a first portion configured to receive an elongated central member;
a second portion configured to receive the elongated central member, a length of the cervical seal corresponding to a distance between the first portion and the second portion along the elongated central member; and
a membrane coupled to the first portion and the second portion and extending along the length of the cervical seal;
wherein the first portion is configured to be movable from a first position along the elongated central member to a second position along the elongated central member, so as to change the length of the cervical seal; and
the membrane is configured so that a radial profile of the membrane increases in response to increasing the length of the cervical seal, so as to provide a conformable cervical seal.

2. The cervical seal of claim 1, wherein the cervical seal is further configured to lock the first portion to the elongated central member.

3. The cervical seal of claim 2, wherein the elongated central member has a plurality of grooves configured to receive a locking portion of the first portion that is movable.

4. The cervical seal of claim 1, wherein the second portion is affixed to the elongated central member, the length the cervical seal being adjustable in response to a movement of the first portion along the elongated central member.

5. The cervical seal of claim 1, wherein the first portion includes a flange having a diameter larger than a diameter of the second portion.

6. The cervical seal of claim 5, wherein the membrane extends from an outer edge of the flange to an outer edge of the second portion.

7. The cervical seal of claim 5, wherein the flange is spring-loaded.

8. The cervical seal of claim 5, wherein the diameter of the flange is larger than a diameter of a cervical anatomy, the flange being configured to provide a physical reference from the cervical seal to the cervical anatomy.

9. The cervical seal of claim 1, wherein the second portion includes a contoured tip configured to locate an internal os of a cervix.

10. The cervical seal of claim 9, wherein the first portion includes a flange having a diameter larger than a diameter of an external os of the cervix so as to locate the external os, the cervical seal further being configured to provide a reference for a measurement of a cervical canal length of the cervix.

11. The cervical seal of claim 1, wherein the membrane is one of a webbing, a sealed balloon and an inflatable balloon.

12. The cervical seal of claim 1, wherein the membrane is backed by one of a conformable structure, a spring-loaded structure, a conformable foam and a spring-loaded foam.

13. The cervical seal of claim 1, wherein the first portion, the second portion and the membrane are made of an elastomeric material, the cervical seal further including a rigid substructure disposed beneath at least one of the first portion and the second portion.

14. The cervical seal of claim 1, wherein the membrane has an accordion shape including a plurality of alternating inner and outer annular rims.

15. The cervical seal of claim 14, wherein the membrane is tapered along at least a portion of the length of the cervical seal, a respective diameter of each annular rim of the plurality of alternating inner and outer annular rims changing along at least the portion of the length of the cervical seal.

16. The cervical seal of claim 1, wherein the membrane has a tubular shape, the cervical seal further including a plurality of ribs disposed along a length of the membrane and protruding radially from a surface of the membrane, the plurality of ribs being pliable so as to allow deflecting.

17. The cervical seal of claim 16, wherein a diameter of the plurality of ribs changes along the length of the membrane.

18. The cervical seal of claim 1, wherein the membrane has a tubular shape, the cervical seal further including a helical rib disposed around the membrane and along a length of the membrane.

19. The cervical seal of claim 18, wherein the cervical seal further includes a longitudinal rib disposed along the length of the membrane and coupled to a plurality of edges of the helical rib.

20. The cervical seal of claim 19, wherein the longitudinal rib is tapered and a diameter of the plurality of edges of the helical rib changes along the length of the membrane.

21. The cervical seal of claim 1, wherein the membrane has a variable durometer along the length of the cervical seal.

22. A cervical seal having an adjustable length, comprising:
a first portion configured to receive an elongated central member;
a second portion configured to receive the elongated central member, a length of the cervical seal corresponding to a distance between the first portion and the second portion along the elongated central member; and
a membrane having a first end coupled to the first portion and a second end coupled to the second portion, the membrane extending along the length of the cervical seal;
wherein the first portion is configured to be movable from a first position along the elongated central member to a second position along the elongated central member, so as to change the length of the cervical seal; and
the first portion having a first diameter and the second portion having a second diameter being configured to remain unchanged in response to increasing the length of the cervical seal.

23. A method of sealing a cervical canal, comprising:
inserting an adjustable length cervical seal into the cervical canal;
increasing a length of the adjustable length cervical seal; and
increasing a radial profile of the adjustable length cervical seal in response to increasing the length.
